(19) United States
(12) Patent Application Publication
(10) Pub. No.: US 2024/0197174 A1
(43) Pub. Date: Jun. 20, 2024

(54) OCULAR HEALTH INDEX DETERMINATION AND USAGE

(71) Applicant: BALANCE OPHTHALMICS, INC., Sioux Falls, SD (US)

(72) Inventors: John Berdahl, Sioux Falls, SD (US); Gary Berman, Laguna Beach, CA (US); Enrico Brambilla, Irvine, CA (US)

(21) Appl. No.: 18/287,275

(22) PCT Filed: Apr. 27, 2022
(86) PCT No.: PCT/US2022/026495

§ 371 Date: Oct. 17, 2023

Related U.S. Application Data
(60) Provisional application No. 63/180,758, filed on Apr. 28, 2021.

(51) Int. Cl. A61B 3/16; A61F 9/00; A61F 9/02; G16H 20/17

(52) U.S. Cl. CPC A61B 3/16; A61F 9/0017; A61F 9/029; G16H 20/17

(57) ABSTRACT

Eye health of a patient can be monitored and various ocular conditions treated such as using information about an intraocular pressure (IOP) characteristic of the patient, information about a blood pressure (BP) characteristic of the patient, and information about a patient therapy, such as from a patient treatment system. In an example, a quantitative ocular health index can be determined for a patient based on information about an IOP characteristic, a BP characteristic, and the patient therapy.

### CLAIM OF PRIORITY

This application is related to and claims priority to U.S. Provisional Application No. 63/180,758, filed on Apr. 28, 2021, and entitled “Ocular Health Index Determination and Usage,” the entirety of which is incorporated herein by reference.

### BACKGROUND

Glaucoma is a chronic condition that causes blindness and predominantly affects elderly members of the population. While glaucoma is not reversible, its progression can be stopped or slowed with treatment. Various existing glaucoma treatments include eye drop medications and surgeries.

Eye drop medication (e.g., prostaglandins, beta blockers, carbonic anhydrate inhibitors, or alpha agonists) is typically the first treatment option since it can be effective for many patients and can have relatively low complication rates. Still, eye drop medications can be ineffective for a significant number of patients. There is also a significant problem with patient compliance in maintaining an eye drop medication regimen on a regular basis. Since glaucoma is a chronic condition, glaucoma patients generally need to take their eye drop medication for the rest of their lives. It has been estimated that up to 50% of glaucoma patients prescribed with an eye drop medication fail to successfully administer their drops on a regular basis. This failure can be due to forgetting, difficulty getting the drops into the eyes, reluctance to take long-term medications, or unhappiness with certain side effects. The side effects can include, for example, redness of the eyes, eyelash growth, inflammation, orbital fat atrophy, discoloration of the iris or surrounding periorbital tissues, exacerbation of COPD or asthma, inhibition of corneal endothelial pump function, exacerbation of corneal edema, or stinging upon instillation.

Various surgical options are available, such as laser trabeculoplasty, trabecular meshwork stents, suprachoroidal stents, subconjunctival stents or trabeculectomy or glaucoma tube shunts. Such surgical treatments are typically the second treatment option for glaucoma patients. Surgical options are more invasive and can have higher complication and morbidity rates as compared to eye drop medication treatments.

### BRIEF SUMMARY

The present inventor has recognized that a problem to be solved includes monitoring ocular disease progression and quickly adapting treatment in correspondence with disease progression. The present inventor has recognized that a solution can include an ocular health management system for monitoring and treating ocular disease progression over time. In an example, the solution can include or use a system comprising an intraocular pressure (IOP) sensor, a blood pressure (BP) sensor, and a patient treatment system. The patient treatment system and one or more of the sensors can operate automatically or can be operated by a patient. In an example, the solution can further include a processor circuit configured to determine an ocular health index for a patient based on IOP information from the IOP sensor, BP information from the BP sensor, and information about a therapy provided to the patient by the patient treatment system. In an example, the ocular health index can represent a disease state or disease progression. The ocular health index can be useful to clinicians and caregivers to monitor disease progression and patient response to therapy.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced. In the drawings, which are not necessarily drawn to scale, like numerals may describe similar components in different views. The drawings illustrate generally, by way of example, but not by way of limitation, various embodiments discussed in the present document.

FIG. 1 illustrates an ocular health management system in accordance with one example.

FIG. 2 illustrates a side cross section view of an eye in accordance with one example.

FIG. 3 illustrates a goggle assembly in accordance with one example.

FIG. 4 illustrates a cross section view in accordance with one embodiment.

FIG. 5 illustrates a first method for evaluating a patient status associated with an eye health of a patient in accordance with one embodiment.

FIG. 6 illustrates a second method for evaluating a patient status associated with an eye health of a patient in accordance with one embodiment.

FIG. 7 illustrates a third method for evaluating a patient status associated with an eye health of a patient in accordance with one embodiment.

FIG. 8 illustrates a fourth method for evaluating a patient status associated with an eye health of a patient in accordance with one embodiment.

FIG. 9 illustrates a fifth method in accordance with one embodiment.

FIG. 10 illustrates generally a diagram of a machine in the form of a computer system within which a set of instructions may be executed for causing the machine to perform any one or more of the methods discussed herein.

### DETAILED DESCRIPTION

Ocular health can be monitored using various systems, methods, or techniques. Various eye-related diseases, such as glaucoma, can have relatively long onset periods. If an eye-related disease such as glaucoma is identified early, it can be possible to monitor disease progression, such as to enable better decision making by clinicians and patients regarding therapy. For example, while glaucoma is not reversible, its progression can be stopped or slowed with treatment directed to reducing intraocular pressure (IOP). In an example, IOP treatment can include various non-invasive methods or methods that do not rely on drug therapies, such as eye drop medications. Non-drug therapy can be important due to problems with patient drug therapy compliance, or ineffectiveness of eye drop medications, or because of actual or potential complications or morbidity rates associated with surgical solutions.

In an example, systems and methods discussed herein can include, or use, or can be used to determine an ocular health index for a patient. An ocular health index can be an objective measure or metric associated with a patient status and indicative of a patient ocular or optical well-being. In an example, an ocular health index can include or refer to a metric that indicates a physiologic status of an eye, an optic nerve, or other structures or features appurtenant to the eye or vision or vision processing.

In an example, the ocular health index can be based on an IOP characteristic of a patient, such as at a particular instant in time or over a particular duration. In an example, the ocular health index can be based on a change in an IOP characteristic of a patient over time. In an example, the ocular health index can be based on, or can be used to inform, a patient therapy. For example, the ocular health index can represent a patient responsiveness or non-responsiveness to a particular therapy, such as a drug therapy, a non-invasive ocular therapy, a surgery, or other therapy.

In an example, the ocular health index can be provided at least in part based on information from a patient treatment system. In an example, the patient treatment system can include or use a goggle or goggles configured to fit over one or both of a patient's eyes to thereby provide a substantially airtight cavity that is bounded in part by an eye or eyes. The goggle can include means to change a pressure inside one or more cavities of the goggle or goggle. The goggle or goggles can seal against a patient's skin, such as at or around a perimeter of a patient eye socket. When the means to alter pressure is actuated, a pressure differential from atmospheric pressure, or gauge pressure, can be created and maintained inside the one or more goggle cavities and, thus, can change an effective pressure on one or both of the eyes. The gauge pressure is positive for pressures above atmospheric pressure, and negative for pressures below it. Absolute pressure, such as inside the goggles, is the sum of gauge pressure and atmospheric pressure.

The cavity pressure can be either increased or decreased relative to atmospheric pressure, depending on a condition being treated (e.g., whether glaucoma is being treated or whether papilledema is being treated). The change in pressure outside of the eye(s) can act to alter a pressure inside the eye(s) (e.g., IOP) through a resulting deflection of the shape of the eye(s), by driving a change in a rate of drainage of eye fluids through a trabecular meshwork, and/or from the pressure difference being directly translated into the eye(s). The pressure can be altered by use of a small compressor or vacuum device (collectively a “pump”) in fluid communication with the one or more cavities of the goggle or goggles.

In an example, the ocular health index can be provided based in part on information from other physiologic sensors or from subjective information received from a patient. For example, information from one or more of a cerebrospinal fluid (CSF) pressure sensor, a blood pressure sensor, an oxygen saturation sensor, an impedance sensor, a weight sensor, or other physiologic sensor can be used. In an example, information received from a patient interface can be used. For example, information about a patient's field of vision, well-being, mood, weight, body mass index, diet, therapy compliance, or other patient-reported information can be used. In an example, the ocular health index can be based in part on information from a treatment system about a therapy compliance of the patient, such as a drug therapy compliance, a CPAP therapy usage or compliance, or other therapy titration information.

In an example, information about the ocular health index can be reported to a clinician or to an automated patient monitoring system. The ocular health index can be reported with or without the underlying data or information on which the ocular health index is based. For example, the ocular health index can be reported with or without the patient's corresponding IOP, weight, drug therapy compliance, or other information.

In an example, the ocular health index can be based in part on an ocular perfusion pressure (OPP). An OPP is generally defined as a mean ocular perfusion pressure (MOPP) that is a function of diastolic blood pressure (DBP), systolic blood pressure (SBP), and IOP. For example, MOPP can be expressed as MOPP=⅔ [DBP+⅓(SBP−DBP)]−IOP.

In an example, the systems and methods discussed herein can be used to ensure compliance with various Current Procedural Terminology (CPT) codes. CPT codes can be used to report various medical, surgical, or diagnostic services to physicians, health insurance companies or accreditation organizations. In an example, systems and methods discussed herein can be used for remote physiologic monitoring or treatment management services, such as on a periodic (e.g., monthly, weekly, etc.) basis. In an example, the systems and methods can be configured to help facilitate interactive communication between the patient and clinician (or other caregiver) such as at a frequency or interval designated per the CPT code. The interactive communication can include, for example, phone, text, or email-based communications, and can occur, for example, at least once per month, per week, per day, such as can be determined on a case-by-case basis.

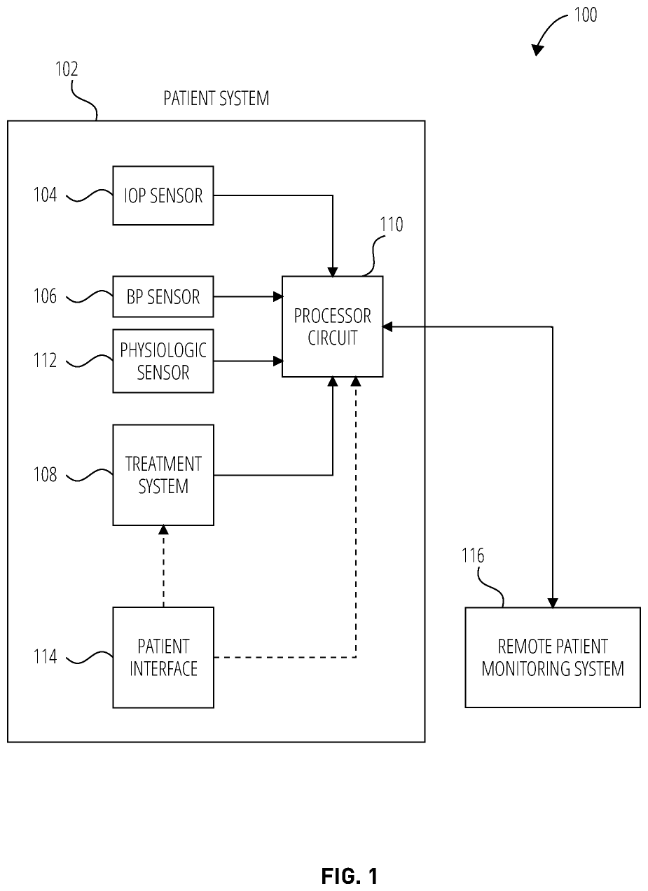

FIG. 1 illustrates generally an example of an ocular health management system 100. The ocular health management system 100 can include a patient system 102, such as can be communicatively coupled to a remote patient monitoring system 116. In an example, the patient system 102 can be coupled with the remote patient monitoring system 116 using a wired or wireless data communication bus, such as can include or use the Internet, a cellular network, or one or more other networks for communicating data in a unidirectional or bidirectional manner.

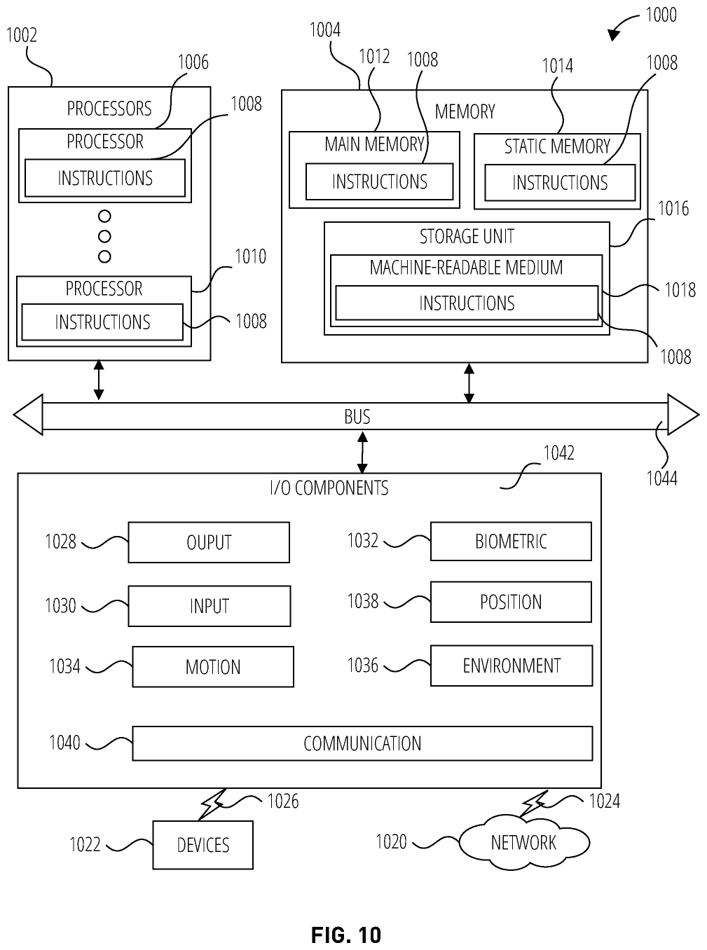

In the example of FIG. 1, the patient system 102 can include an IOP sensor 104, a BP sensor 106, a physiologic sensor 112, and a treatment system 108. The IOP sensor 104, the BP sensor 106, the physiologic sensor 112, and the treatment system 108 can have respective dedicated processors or processor circuits, or can be coupled to a common central processor or processor circuit 110. For simplicity, the example of FIG. 1 shows an embodiment with a central processor circuit 110. The example of FIG. 1 further includes a patient interface 114, such as can be coupled to one or both of the treatment system 108 and the processor circuit 110. In an example, the processor circuit 110 can include or use one or more of the processors or processor circuits discussed herein in FIG. 10. Other processors, ASICs, or circuitry can similarly be used.

The IOP sensor 104 can include a physiologic sensor that is configured to measure intraocular pressure in a patient eye, such as using tonometry, using transpalpebral techniques, using contact lens-based systems, or using implanted devices. While tonometers are not invasive, they are generally expensive, not portable, and require a skilled operator. Accordingly, as a practical matter, it can be difficult to use a tonometer to effectively monitor IOP in a patient's eye over time, such as over the course of a day, a week, or longer. Since IOP can vary significantly over relatively short periods of time, sparse pressure measurements may not provide a complete or accurate view of a patient's risk for glaucoma. Implanted devices or contact lens-based systems can, in some examples, include other sensors such as for monitoring glucose (e.g., as measured from the aqueous humor in the eye), heart rate, blood pressure, oxygen saturation, or other physiologic status information about the patient.

The IOP sensor 104 can be configured to provide information about an intraocular pressure (IOP) characteristic of the patient to the processor circuit 110. The information can include, among other things, an absolute pressure value (e.g., in mmHg or other unit), a change in pressure over time, a pressure value relative to a reference, or other information about the IOP of the patient.

In an example, the BP sensor 106 can include a physiologic sensor that is configured to measure a patient blood pressure. The BP sensor 106 can include an arm cuff-based measurement system, an impedance-based system, or one or more other invasive, implanted, or non-invasive means for measuring diastolic and systolic pressure in a patient blood vessel.

The BP sensor 106 can be configured to provide information about a blood pressure (BP) characteristic of the patient to the processor circuit 110. The information can include, among other things, an absolute pressure value (e.g., in mmHg or other unit), a change in pressure over time, a pressure value relative to a reference, or other information about the BP of the patient.

The physiologic sensor 112 can include a sensor configured to sense one or more physiologic parameters about a patient or a patient health status. In an example, the physiologic sensor 112 can include an invasive (e.g., implanted or partially implanted) or non-invasive system or device, or a wearable device. The physiologic sensor 112 can be configured to monitor one or more of a body impedance characteristic, a blood chemical concentration, a temperature, a pH level, a sleep duration or sleep quality, an acoustic signal, a blood oxygenation, an airflow, or one or more other physiologic aspects or indications of physiologic function of the patient.

In an example, one or more of the IOP sensor 104, the BP sensor 106, or the physiologic sensor 112 can be responsive to instructions from the processor circuit 110. For example, the processor circuit 110 can be configured to coordinate physiologic sensing or measurement activity by one or more of the sensors. The sensors can be configured to measure physiologic information about a patient concurrently or at different times. For example, the processor circuit 110 can coordinate receiving IOP information from the IOP sensor 104 and BP information from the BP sensor 106 at substantially the same time such as to provide a caregiver a more comprehensive or complete physiologic picture of a monitored patient.

In an example, the treatment system 108 can include various systems such as for treating one or more diseases or for providing one or more therapies to a patient. In an example, the treatment system 108 can include or use a goggle-based glaucoma treatment or therapy system, such as can be configured to provide a gauge pressure at or adjacent to a patient eye to thereby adjust an IOP of the eye. In an example, the treatment system 108 can include or use an automated or semi-automated drug delivery system, such as can be configured to provide a drug or chemical therapy to a patient, such as by delivering such drug or chemical directly to the patient's body (e.g., intravenously or otherwise) or by tracking dispensing of a drug or chemical. For example, the treatment system 108 can include an eye drop delivery system configured to count or track a number of drops of volume of drop liquid dispensed from the treatment system 108. In an example, the treatment system 108 can include a system configured to treat obstructive sleep apnea or central sleep apnea, such as a continuous positive airway pressure (CPAP) system or a bilevel positive airway pressure (BiPAP) system, or other system. The treatment system 108 can be configured to provide information about a treatment, therapy, patient response, or other information to the processor circuit 110.

The remote patient monitoring system 116 can include a system or network of systems of devices configured to enable clinicians, healthcare providers, patients, and others, to remotely collect and review physiologic information or other health status information from or about a patient. In some examples, one or more sensors associated with a patient can be in data communication with a remote system that is accessible by the patient's healthcare providers. In an example, a communicator or base station can be provided in a patient's home. The base station can include an interface configured to receive physiological data from the sensors, and can include a network interface that can upload the physiological data to a remote server, such as can be accessed by one or more of the patient's healthcare providers. The network can include or use a data communication network such as the Internet, and can be wired or wireless. In some examples, use of remote patient monitoring systems can correlate to reduced healthcare costs and improved patient outcomes.

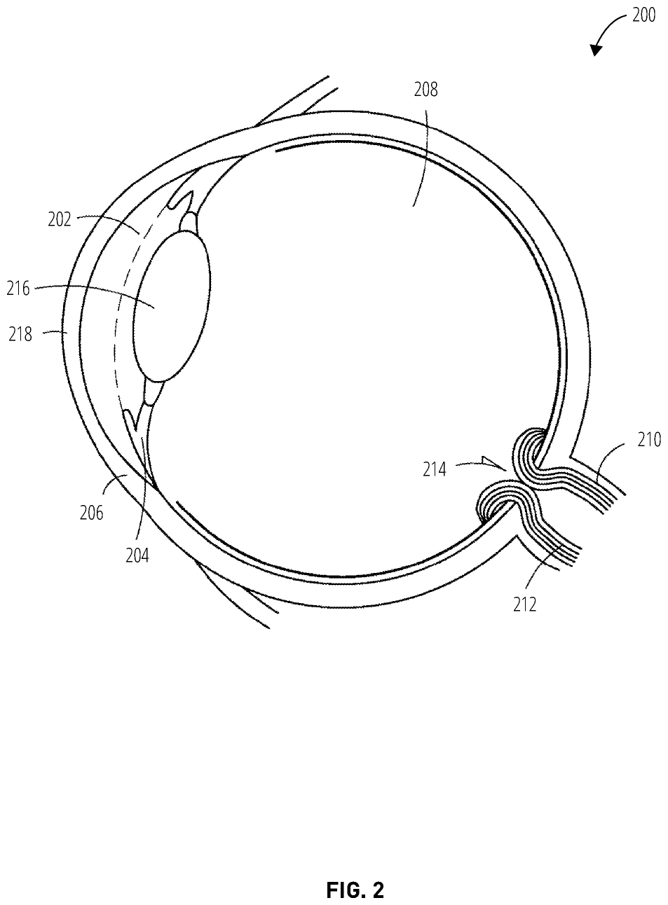

FIG. 2 illustrates generally a side cross section view of an eye 200. The side cross section view of an eye 200 comprises an anterior chamber 202, a ciliary muscle 204, a trabecular meshwork 206, a vitreous humor 208, a cerebrospinal fluid 210, an optic nerve 212, a lamina cribrosa 214, a lens 216, and a cornea 218.

The example of FIG. 2 shows a cornea 218 and an anterior chamber 202 or aqueous chamber behind the cornea 218. The anterior chamber 202 is the primary reservoir of aqueous humor inside the eye. Located behind the anterior chamber 202 is a lens 216, and behind and around the lens 216 is the ciliary muscle 204 or ciliary processes where aqueous humor is produced. The ciliary muscle 204 is roughly in the same plane as the lens 216 and is positioned around the perimeter of the lens 216. A trabecular meshwork 206, Schlemm's canal, and anterior ciliary veins are all located below the anterior chamber 202 at the bottom of the cornea 218. This is the primary pathway for aqueous humor to exit the eye.

Behind the lens 216 and the ciliary muscle 204 is the posterior chamber of the eye filled with vitreous humor 208. The vitreous humor 208 is distinct from the aqueous humor, but the separation between the chambers holding such fluids is elastic and, as such, the pressures of the two different humor fluids can be equal or approximately equal. An optic nerve 212 connects to the back of the eye. A lamina cribrosa 214 is a membrane over the junction of the optic nerve 212 and the eye. Cerebrospinal fluid 210 baths the optic nerve 212 behind the lamina cribrosa 214 so that the lamina cribrosa 214 is influenced on one side by a pressure of the cerebrospinal fluid 210 and on the other side by the IOP.

During normal function of an eye, aqueous humor is produced inside the eye by the ciliary processes in an anterior segment of the eye. As aqueous humor is steadily produced, a like amount of fluid must exit from the anterior chamber 202 of the eye to maintain a balanced eye pressure. The aqueous humor can exit the anterior chamber 202 by one or both of two main pathways. Some is reabsorbed by the uveoscleral outflow tract around the ciliary muscle 204. Some exits the eye though the trabecular meshwork 206, a porous region in the front of the eye located between the cornea 218 and iris insertion. The aqueous fluid that exits via the trabecular meshwork flows through Schlemm's canal into the anterior ciliary veins. In an example, systems and methods discussed herein can be used to change an IOP in one or both of a patient's eyes, for example, by altering a flow rate of aqueous humor across the trabecular meshwork 206, Schlemm's canal, and the anterior ciliary vein pathway.

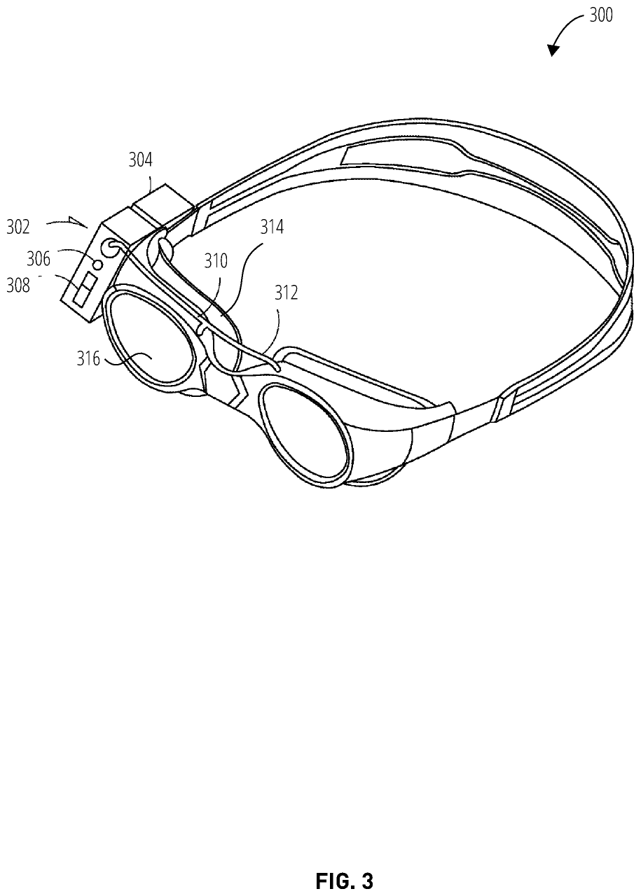

FIG. 3 illustrates generally a perspective view of an example of a goggle assembly 300. In an example, the treatment system 108 from the example of FIG. 1 can include or use the goggle assembly 300. In the example of FIG. 3, the goggle assembly 300 comprises a pump 302, a battery 304, a dial 306, a switch 308, a manifold 310, a tube 312, a gasket 314, and a goggle lens 316. In an example, the goggle assembly 300 can be used to change an IOP in one or both a patient's eyes.

The goggle assembly 300 can include a goggle or goggles that provide one or more eye cavities when the goggle assembly 300 is worn by a patient. The body of the goggle assembly 300 can be relatively rigid and made of an impermeable plastic so that it can maintain a differential pressure inside the cavity or cavities. In an example, a goggle lens 316 portion of the goggle assembly 300 can be transparent to allow a patient wearing the goggle or goggles to see through the cavity. In an example, a gasket 314 can include a compressible material provided around a perimeter of the goggle body, that can create a seal between edges of the goggle or goggles and the patient's skin or other tissue around the eyes. The seal material can be a foam, rubber or plastic material held in close contact with the skin by a strap or headband configured to be worn around the head.

The example of FIG. 3 illustrates a pressure control mechanism can be used with or mounted to the goggle assembly 300. The pressure control mechanism can include a pump 302 such as can be configured as a pressure or compressor pump, or vacuum pump. The pump 302 can be configured to be reversible so that the same goggle assembly 300 can be used for raising or lowering a pressure in the goggle cavity. The pump 302 can be in fluid communication with the cavity portion of the goggle assembly 300 using a manifold 310 and tube 312. The pump 302 can be powered by a battery 304. In an example, the pump 302 can be actuated by means of a switch 308 (e.g., on/off switch). A set point or range for a target goggle cavity pressure can be set or adjusted using a dial 306 or other input means. The adjustable pressure set point or range can control a pressure in a closed loop manner, such as when a pressure sensor configured to monitor the cavity pressure is used. Other means for controlling or adjusting pressure can include a speed control for the pump 302, or control for one or more vents that can be provided in the goggle assembly 300, such as in the manifold 310 or in one or more of the cavities.

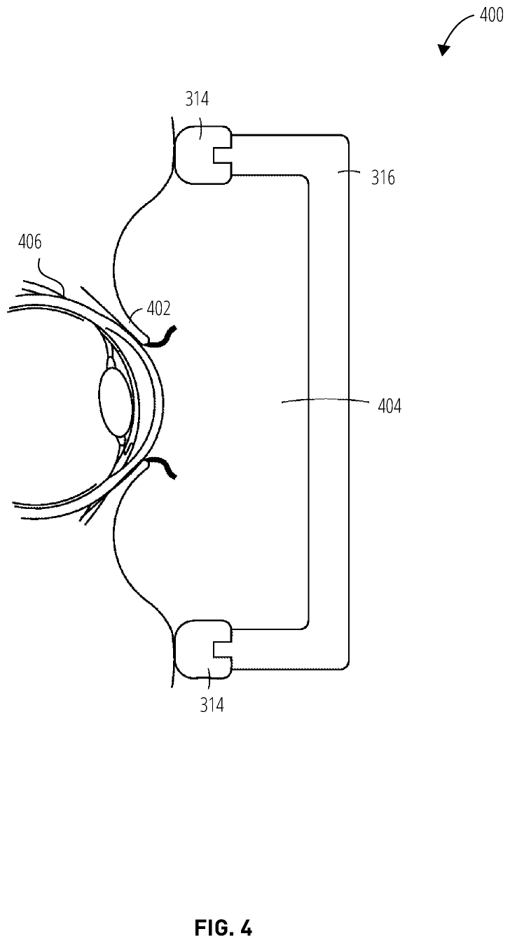

FIG. 4 illustrates generally an example of a cross section view 400 of the goggle assembly 300 fitted to a patient eye 406. The cross section view 400 includes a gasket 314, a goggle lens 316, an eye lid 402, a cavity 404, and a patient eye 406. In the example of FIG. 4, the cavity 404 can be provided between the goggle lens 316 and the patient eye 406.

A body portion of the goggle assembly 300 can be relatively rigid and made of an impermeable material, such as plastic or other polymer, so that the goggle assembly 300 can achieve and maintain a differential pressure, or gauge pressure, inside the cavity 404. The gasket 314 can be positioned around a perimeter of the goggle body, which can be configured to provide a substantially airtight seal between edges of the goggle and the patient's skin around the eyes. In an example, the gasket 314 is configured to contact a portion of the patient's face that is near or adjacent to the eye lid 402.

A pressure in the cavity 404 can be altered by various components of the assembly. The altered air pressure in the cavity 404 can act on an area in front of the patient eye 406. In an example, the altered air pressure can be a reduced air pressure for patients with glaucoma, or patients with high IOP, so that the pressure acts to decrease the patient's IOP. In an example, the altered pressure can be an increased air pressure for patients with optic disk edema, or swelling of the lamina cribrosa. In an example, a pressure in the cavity 404 can be changed or updated over time such as to different target pressures.

Although the goggle assembly 300 as illustrated is configured for use against the patient eye 406, the assembly can be otherwise configured to be worn elsewhere on the body, such as to provide a cavity and gauge pressure at various other tissue locations. For example, an assembly can be configured to provide a cavity in a rectangular or oval shape and can be configured to be positioned against the back of a patient's neck. The assembly can thus be provided to apply a gauge pressure to skin tissue in the neck, such as can be useful for treating headaches or other discomfort. The assembly can be configured to provide cavities of various different shapes and sizes such as for use elsewhere on the body. In an example, the assembly can be configured to provide a cupping therapy. Cupping therapy is used to change blood flow or supply to a relatively small tissue area. The therapy is used by various athletes to aid in recovery or healing of muscle tissue. In an example, the assembly can include or use an integrated heating mechanism to heat or cool the cavity or the air therein.

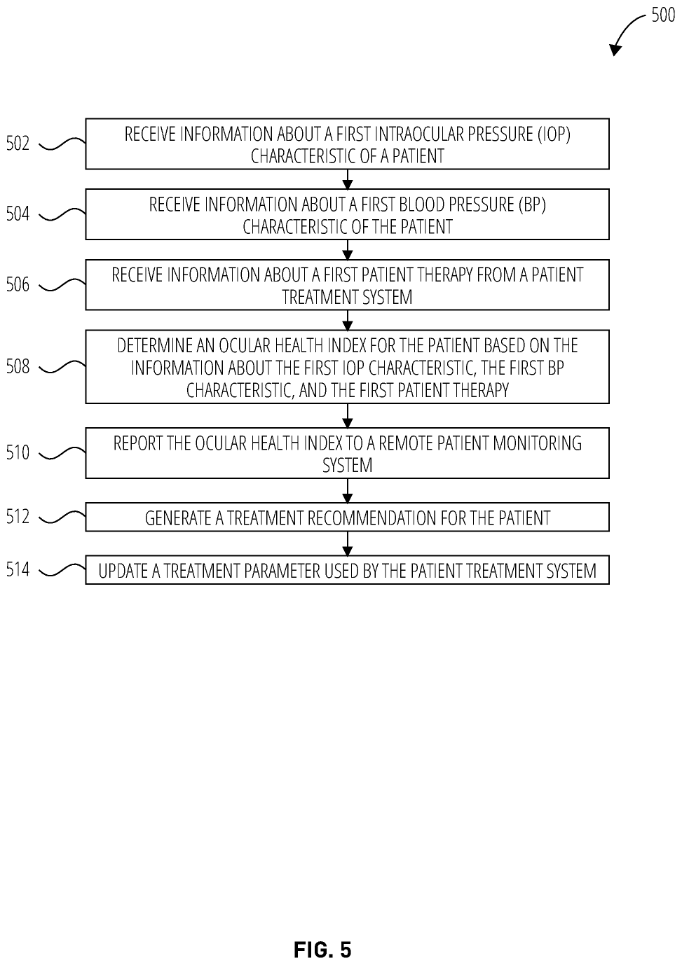

FIG. 5 illustrates generally an example of a first method 500 that can include determining an ocular health index for a patient, such as using the ocular health management system 100. At block 502, the first method 500 can include receiving information about a first IOP characteristic of a patient. Block 502 can include or use information from the IOP sensor 104. In an example, block 502 can include using the processor circuit 110 to receive the information about the first IOP characteristic of the patient.

At block 504, the first method 500 can include receiving information about a first BP characteristic of the same patient for whom the first IOP characteristic was received at block 502. Block 504 can include or use information from the BP sensor 106. In an example, block 504 can include using the processor circuit 110 to receive the information about the first BP characteristic of the patient.

At block 506, the first method 500 can include receiving information about a first patient therapy from a patient treatment system. In an example, block 506 can include receiving information about a therapy or treatment provided to the patient using the treatment system 108. The information about the first patient therapy can include, by way of example and not limitation, information about a glaucoma therapy, an apnea therapy, a dry eye therapy, a headache or migraine therapy, or other therapy configured to treat or cure a patient disease. The information about the therapy or treatment can include information about a treatment duration, a drug titration (e.g., a delivered amount or prescribed amount of a drug), a treatment frequency, a patient-reported or automatically-reported patient adherence to a therapy regimen, a patient response to a particular therapy, or other information about a therapy.

In an example, the information about the therapy or treatment can be received at block 506 from a patient, such as using the patient interface 114. In an example, the patient interface 114 includes a graphical user interface upon which the patient or user, or clinician or other caregiver, can provide information about drug administration. In an example, the patient interface 114 can be configured to remind or prompt the patient to take a drug or administer a drug therapy, or can be configured to remind or prompt the patient to report a drug administration event.

At block 508, the first method 500 can include determining an ocular health index for the patient. Block 508 can include using the processor circuit 110 to process information from at least the IOP sensor 104 and the BP sensor 106 and, in response, generate the ocular health index. In an example, the processor circuit 110 can use information about the therapy or treatment, such as received at block 506, to determine the ocular health index.

In an example, the ocular health index determined at block 508 can include a quantitative indication of a disease state, disease progression, or a risk of disease progression, such as glaucoma or dry eye progression. In an example, the ocular health index can be a predictive index or metric associated with a trend in ocular patient health. In an example, block 508 can include or use glaucoma-related data, such as can be acquired or determined using the ocular health management system 100 or other system or device over time, to determine the ocular health index. In an example, block 508 includes using physiologic information from a patient from diurnal monitoring periods, nocturnal monitoring periods, or both, to determine the ocular health index.

In an example, the ocular health index can be determined at block 508 using clinician-generated data or information about a patient. In an example, the clinician-generated data or information can be provided to the remote patient monitoring system 116 by a clinician, and in response, the remote patient monitoring system 116 or the processor circuit 110 can use the data or information to determine the ocular health index.

In an example, determining the ocular health index at block 508 can include or use information from the treatment system 108. For example, information from the goggle assembly 300 can be used. The information can include pressure information, such as information about a magnitude or duration of pressure applied to a patient eye using the goggle assembly 300. The information from the treatment system 108 can include an average or mean pressure, such as an average daily or nightly pressure applied to a patient by or using the goggle assembly 300.

At block 510, the first method 500 can include reporting the ocular health index to a remote system or device, such as to the remote patient monitoring system 116. In an example, block 510 includes receiving the ocular health index as-determined at block 508, such as using the processor circuit 110, and communicating the ocular health index to the remote patient monitoring system 116 using a wired or wireless data communication system.

At block 512, the first method 500 can include generating a treatment recommendation for the patient based on the ocular health index. In an example, block 512 includes receiving an input or instructions, indicative of a treatment recommendation, from a clinician at the remote patient monitoring system 116. In an example, block 512 includes automatically determining the treatment recommendation at the remote patient monitoring system 116 based on one or more algorithms or machine learning-based decision trees that can use the ocular health index information, such as alone or together with other physiologic status information about a patient or patient population. For example, block 512 can include comparing an ocular health index for a first patient with indexes from a population of similar patients and, based on information about outcomes of the similar patients, providing the treatment recommendation that is determined to be most likely to successfully treat the first patient. Similar patients or patient groups can be identified automatically by the remote patient monitoring system 116 or can be manually designated, such as based on age, race, diet, gender, other health status information or disease state information, or other patient attribute.

At block 514, the first method 500 can include updating a treatment parameter used by the patient treatment system, such as the treatment system 108. In an example, block 514 can include receiving the treatment recommendation generated at block 512 and, in response, updating or changing a therapy parameter used by the treatment system 108. The therapy parameter can influence, for example, a pressure or prescribed frequency of use of the goggle assembly 300 for a patient. The therapy parameter can influence, for example, a drug therapy, a tissue pressure therapy, a CPAP or BiPAP therapy, an electrostimulation therapy, or other patient therapy.

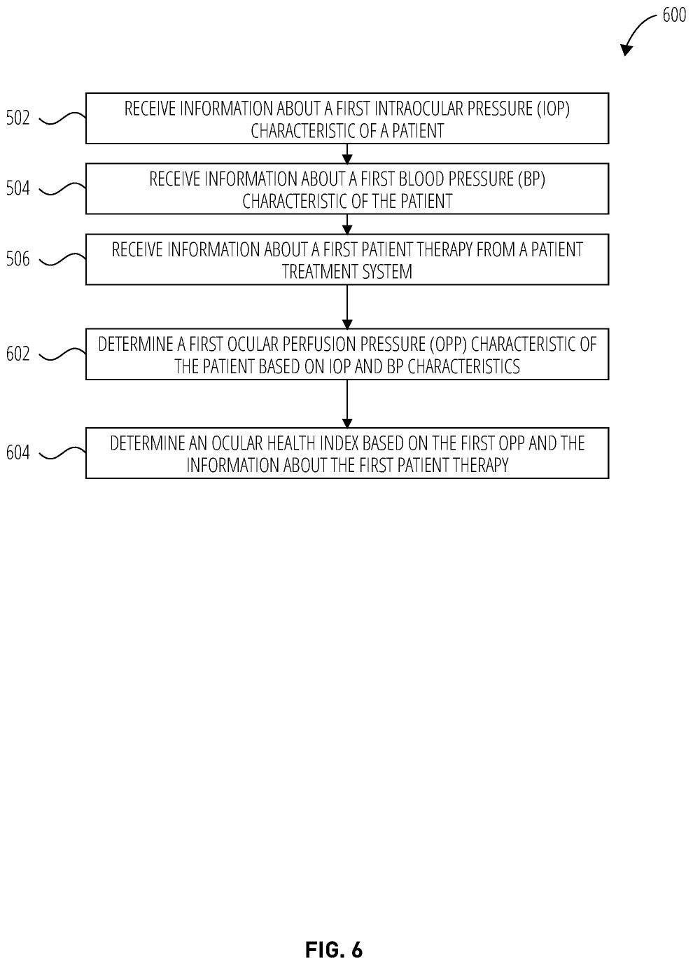

FIG. 6 illustrates generally an example of a second method 600 that can include using an ocular perfusion pressure (OPP) of a patient to determine an ocular health index of the patient. The second method 600 can include or use the same or similar steps as described in the first method 500 at block 502, block 504, and block 506. At block 602, the second method 600 can include determining an ocular perfusion pressure (OPP) characteristic of the patient. An OPP characteristic can be based on at least an IOP characteristic and BP characteristic.

For example, an OPP, or mean ocular perfusion pressure (MOPP), can be a function of diastolic blood pressure (DBP), systolic blood pressure (SBP), and IOP. For example, MOPP can be expressed as MOPP=⅔ [DBP+⅓(SBP−DBP)]−IOP. In an example, average or mean information about IOP or BP can be used to provide a MOPP that indicates a patient health status over time rather than at a particular time or instant. For example, IOP and/or BP information can be acquired over the course of a day, or over the course of a specified diurnal period (e.g., 4 hours, or more or less), or a specified nocturnal period (e.g., 1 or more hours, or more or less). In an example, acquisition of IOP and/or BP information for use in determining a MOPP can be triggered based on one or more conditions or events. For example, IOP or BP information can be acquired when REM sleep is detected, or when a CPAP therapy is activated or deactivated, or at a designated time following administration of a drug therapy.

At block 604, the second method 600 can include determining an ocular health index based on the OPP determined at block 602 and based on information about a patient therapy received at block 506. Block 604 can include using the processor circuit 110 to process information about the OPP together with the therapy information to generate the ocular health index. In an example, the ocular health index determined at block 604 can have one or more attributes of the ocular health index described above in the discussion of block 508. For example, the ocular health index determined at block 604 can include a quantitative indication of a disease state, disease progression, or a risk of disease progression. Following block 604, the ocular health index determined using the OPP characteristic can be reported to a remote system or device, such as to the remote patient monitoring system 116.

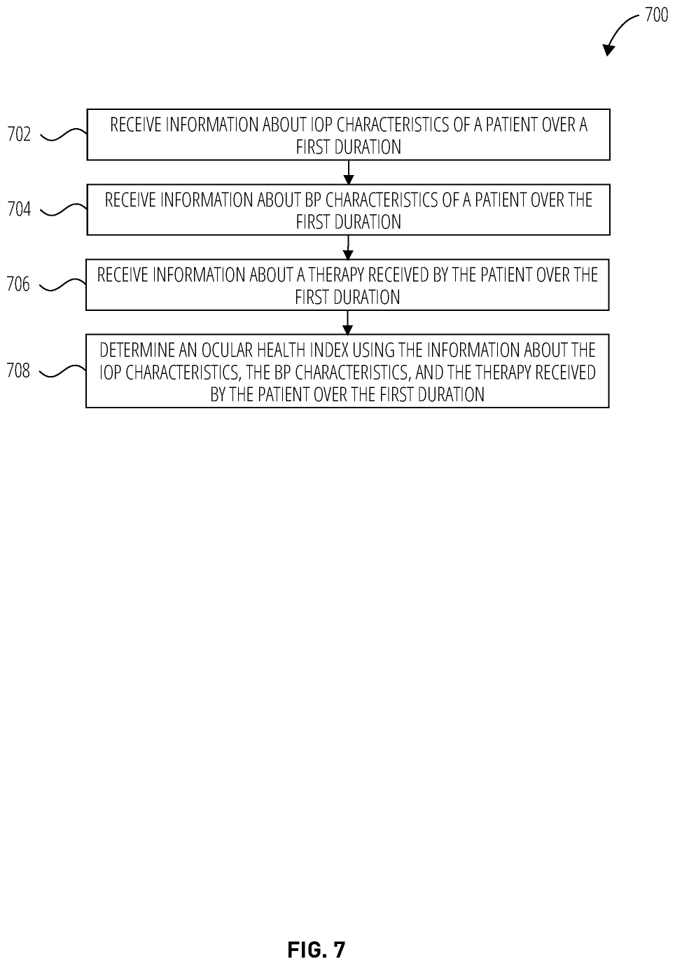

FIG. 7 illustrates generally an example of a third method 700 that can include using sensor information, acquired over time, to determine an ocular health index for a patient. The third method 700 can be performed using one or more components of the ocular health management system 100.

At block 702, the third method 700 can include receiving information about a first IOP characteristic of a patient over time, or over a first duration. Receiving the first IOP characteristic over time can include measuring IOP at multiple discrete instances and determining an average, a moving average, a standard deviation, or other indication of absolute or relative IOP of a patient over a specified duration. The duration can be specified to be of various lengths. For example, the duration can be several seconds, minutes, hours, or days. In an example, the duration can represent like-intervals over multiple times, such as several minutes every hour, or several minutes every day, or several minutes during a diurnal monitoring period, or several minutes during a nocturnal monitoring period. Other durations can similarly be used. Block 702 can include using the IOP sensor 104 to acquire the IOP characteristic information and receiving the IOP characteristic information at the processor circuit 110.

At block 704, the third method 700 can include receiving information about a first BP characteristic over the same first duration. In other examples, the information about the first BP characteristic can be received over a different duration, such as a duration at least partially overlapping with the first duration, or at a different duration than the first duration. Block 704 can include or use information from the BP sensor 106. In an example, block 704 can include using the BP sensor 106 to acquire the BP characteristic information and receiving the BP characteristic information at the processor circuit 110.

At block 706, the third method 700 can include receiving information about a therapy received by the patient over the same first duration. In other examples, the information about the therapy received by the patient can be received over a different duration, such as a duration at least partially overlapping with the first duration, or at a different duration than the first duration. In an example, block 706 can include receiving information about a therapy or treatment provided to the patient using the treatment system 108. Various types of information can be received at block 706, for example, information about a glaucoma therapy, an apnea therapy, a dry eye therapy, a headache or migraine therapy, or other therapy configured to treat or cure a patient disease. The information about the therapy can include information about a treatment duration, a drug titration (e.g., a delivered amount or prescribed amount of a drug), a treatment frequency, a patient-reported or automatically-reported patient adherence to a therapy regimen (e.g., using a monitoring device that is configured to monitor and report drug administration), a patient response to a particular therapy, or other information.

In an example, block 706 can include using the information about the therapy to determine a relationship between therapy administration and a duration of therapy efficacy. For example, block 706 can include cross-checking a time of delivery of a drug (or other therapy) with an expected, or patient-reported, efficacy. In an example, the efficacy information can be determined using patient-reported information, such as via the patient interface 114, or the efficacy can be determined using more objective physiologic information, such as IOP characteristic information (e.g., received at block 702) or BP characteristic information (e.g., received at block 704).

At block 708, the third method 700 can include determining an ocular health index for the patient. Block 708 can include using the processor circuit 110 to process information received from at least the IOP sensor 104 at block 702, from the BP sensor 106 at block 704, and information about the therapy from block 706 and, in response, to generate the ocular health index. As similarly explained elsewhere herein, the ocular health index can include a quantitative indication of a disease state, disease progression, or a risk of disease progression, such as glaucoma or dry eye progression.

In an example, block 708 can include an indication of a therapy efficacy for a patient. For example, block 708 can include using the efficacy information (actual or expected) from block 706 together with the information about the IOP and BP characteristics from blocks 702 and 704 to determine the ocular health index. In an example, the ocular health index can have a value that depends, at least in part, on a measured physiologic responsiveness to the therapy.

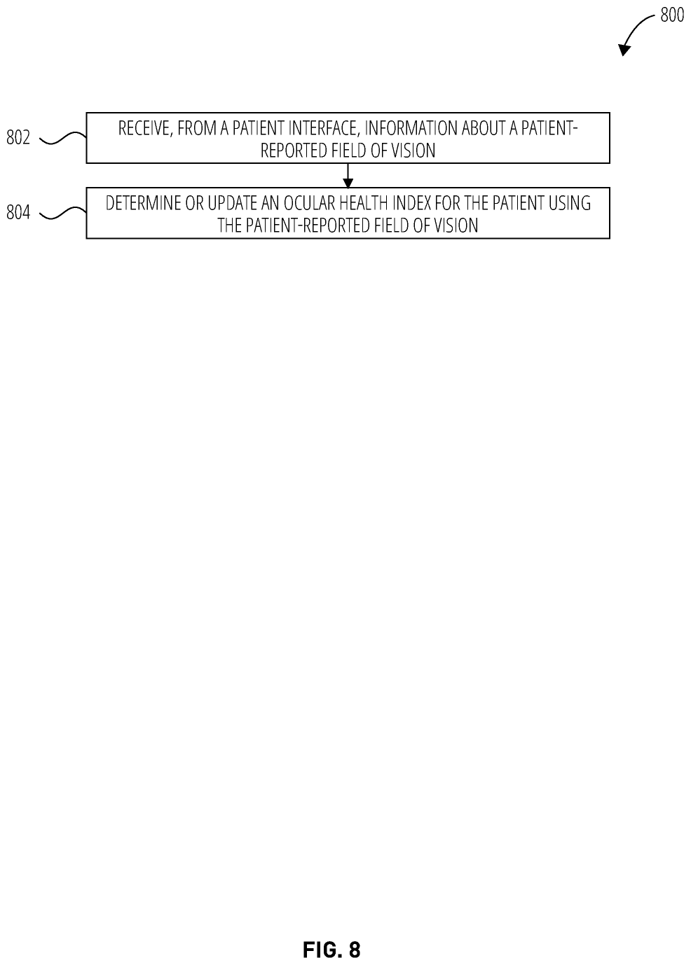

FIG. 8 illustrates generally an example of a fourth method 800 that can include determining or updating an ocular health index. For example, at block 802, the fourth method 800 can include receiving information from an interface or input device, such as the patient interface 114. In the example of FIG. 8, block 802 includes receiving information about a patient-reported field of vision using the patient interface 114. In an example, the patient interface 114 includes a graphical user interface upon which the patient or user, or clinician or other caregiver, can provide information about a visual field available to or experienced by the patient. For example, the patient interface 114 can include means for taking a panoramic photo and displaying the photo on a screen for the patient. The patient can then indicate on the screen the patient's field of vision, with respect to the panoramic view. The patient's field of vision can be quantitatively analyzed, such as to provide an objective indication of the patient's field of vision or change in the patient's field of vision.

In an example, block 802 can include using a virtual reality (VR) headset or other head-mounted system to display visual information to a patient, such as to provide a visual field test to the patient. Patient-reported information about the displayed visual information can be received at block 802. In an example, the headset can include an inexpensive device such as Google Cardboard that can be used with or paired with a patient's mobile device, tablet, or other computer. Using such a headset at block 802 can help enable patients to perform vision tests frequently and on their own. Results can be delivered to the patient in real-time or can be reported to a remote clinician. In an example, the information received at block 802 can be used to help expedite identification or treatment of eye disease or vision loss, and can help reduce incidence of irreversible damage due to untreated disease progression.

At block 804, the fourth method 800 can include determining or updating an ocular health index for the patient using the information received at block 802. That is, block 804 can include using one or more of the processor circuit 110 or the remote patient monitoring system 116 to receive information about the patient's field of view, such as reported by the patient at block 802, and in response updating the ocular health index of the patient. The updated ocular health index can then be provided to a clinician or other caregiver for monitoring or intervention purposes. For example, if the ocular health index is trending in a negative manner indicating a lesser field of vision experienced by the patient, then in response the clinician or caregiver can change a therapy parameter or update a drug therapy for the patient. In an example, one or more therapy parameters can be updated and implemented in response to the change in ocular health index, such as using the treatment system 108 for the patient.

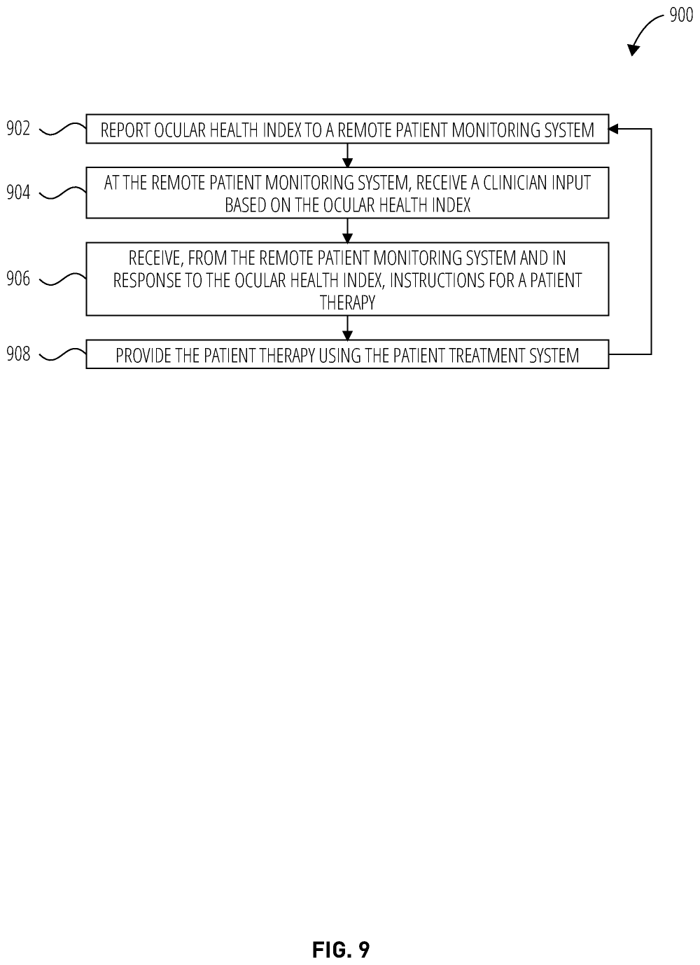

FIG. 9 illustrates generally an example of a fifth method 900 that can include updating a therapy for a patient based on an ocular health index about the patient. At block 902, the fifth method 900 can include reporting an ocular health index to a remote system. For example, block 902 can include using the processor circuit 110 to report or communication a patient ocular health index to the remote patient monitoring system 116.

At block 904, at the remote patient monitoring system 116, information about the ocular health index can be provided to a clinician or other caregiver using a clinician interface. Based on the ocular health index, and optionally based on one or more other criteria, the clinician can provide an input to the remote patient monitoring system 116 indicative of an update for the patient who receives therapy or treatment from the treatment system 108. At block 906, the fifth method 900 can include receiving, at the treatment system 108, instructions for beginning, ending, maintaining, or changing a patient therapy, such as can be provided to the patient using the treatment system 108. In an example, information about the therapy update can be provided to the patient using the patient interface 114. At block 908, the fifth method 900 can include providing the patient therapy to the patient using the treatment system 108 in accordance with the instructions or parameters received at block 906.

Following block 908, the fifth method 900 can include providing therapy to the patient for at least a specified minimum duration or therapy interval. Following the therapy interval, the fifth method 900 can return to block 902 and report a new or updated ocular health index to the remote patient monitoring system 116. In an example, the fifth method 900 can be performed according to a clinician-specified schedule or can be performed on demand. For example, the fifth method 900 can be performed in accordance with timing requirements specified by one or more CPT codes, such as can be updated from time to time depending on patient disease state or best practices.

FIG. 10 is a diagrammatic representation of a machine 1000 within which instructions 1008 (e.g., software, a program, an application, an applet, an app, or other executable code) for causing the machine 1000 to perform any one or more of the methodologies discussed herein can be executed. For example, the instructions 1008 can cause the machine 1000 to execute any one or more of the methods described herein. The instructions 1008 can transform the general, non-programmed machine 1000 into a particular machine 1000 programmed to carry out the described and illustrated functions in the manner described. The machine 1000, or various instances of the machine 1000, can comprise or can be used to implement one or more portions of the ocular health management system 100.

In an example, the machine 1000 can operate as a standalone device or can be coupled (e.g., networked) to other machines or devices or processors. In a networked deployment, the machine 1000 can operate in the capacity of a server machine or a client machine in a server-client network environment, or as a peer machine in a peer-to-peer (or distributed) network environment. For example, one instance of the machine 1000 can comprise some or all of the patient system 102, another instance of the machine 1000 can comprise the patient interface 114, and another instance of the machine 1000 can comprise the remote patient monitoring system 116, and the various instances can be networked together. The machine 1000 can comprise a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1008, sequentially or otherwise, that specify actions to be taken by the machine 1000. Further, while only a single machine 1000 is illustrated, the term “machine” can be taken to include a collection of machines that individually or jointly execute the instructions 1008 to perform any one or more of the methodologies discussed herein. In an example, the instructions 1008 can include instructions stored using a memory circuit, and the machine 1000 can include or use a processor circuit, such as the processor circuit 110, such as can be associated with any one or more of the various blocks, modules, processors, or other processing hardware or software discussed herein.

The machine 1000 can include various processors and processor circuitry, such as represented in the example of FIG. 10 as processors 1002, memory 1004, and I/O components 1042, which can be configured to communicate with each other via a bus 1044. In an example, the processors 1002 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) can include, for example, a first processor 1006 and a second processor 1010 that execute the instructions 1008. The term “processor” is intended to include multi-core processors that can comprise two or more independent processors (sometimes referred to as “cores”) that can execute instructions contemporaneously. Although FIG. 10 shows multiple processors, the machine 1000 can include a single processor with a single core, a single processor with multiple cores (e.g., a multi-core processor), multiple processors with a single core, multiple processors with multiples cores, or any combination thereof.

The memory 1004 can include a main memory 1012, a static memory 1014, or a storage unit 1016, such as can be accessible to the processors 1002 via the bus 1044. The memory 1004, the static memory 1014, and storage unit 1016 can store the instructions 1008 embodying any one or more of the methods or functions or processes described herein. The instructions 1008 can also reside, completely or partially, within the main memory 1012, within the static memory 1014, within the machine-readable medium 1018 (e.g., comprising a non-transitory computer-readable storage medium) within the storage unit 1016, within at least one of the processors (e.g., within a processor's cache memory), or any suitable combination thereof, during execution thereof by the machine 1000.

The I/O components 1042 can include a wide variety of components to receive input, provide output, produce output, transmit information, exchange information, capture measurements, and so on. The specific I/O components 1042 that are included in a particular machine will depend on the type of machine. For example, portable machines such as mobile phones can include a touch input device or other such input mechanisms, while a headless server machine will likely not include such a touch input device. It will be appreciated that the I/O components 1042 can include other components that are not shown in FIG. 10. In various example embodiments, the I/O components 1042 can include output components 1028 and input components 1030. The output components 1028 can include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1030 can include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), physiologic sensor components, and the like.

In an example, the I/O components 1042 can include biometric components 1032, motion components 1034, environmental components 1036, or position components 1038, among other components. For example, the biometric components 1032 include components configured to detect a presence or absence of humans, pets, or other individuals or objects, or configured to detect expressions (e.g., hand expressions, facial expressions, vocal expressions, body gestures, or eye tracking), measure biosignals (e.g., intraocular pressure, cerebrospinal fluid pressure, blood pressure, heart rate, body temperature, perspiration, or brain waves, among others), identify a person (e.g., voice identification, retinal identification, facial identification, fingerprint identification, or electroencephalogram-based identification), and the like. The motion components 1034 can include acceleration sensor components (e.g., accelerometer), gravitation sensor components, rotation sensor components (e.g., gyroscope), and so forth.

The environmental components 1036 can include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that can provide indications, measurements, or signals corresponding to a surrounding physical environment. The position components 1038 include location sensor components (e.g., a GPS receiver component, an RFID tag, etc.), altitude sensor components (e.g., altimeters or barometers that detect air pressure from which altitude can be derived), orientation sensor components (e.g., magnetometers), and the like. Information from any one or more of the I/O components 1042, environmental components 1036, position components 1038, or other components can be used to determine or update an ocular health index for a patient.

The I/O components 1042 can include communication components 1040 operable to couple the machine 1000 to a network 1020 or devices 1022 via a coupling 1024 and a coupling 1026, respectively. For example, the communication components 1040 can include a network interface component or another suitable device to interface with the network 1020. In further examples, the communication components 1040 can include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1022 can be another machine or any of a wide variety of peripheral devices (e.g., a peripheral device coupled via a USB).

Moreover, the communication components 1040 can detect identifiers or include components operable to detect identifiers. For example, the communication components 1040 can include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals), such as can be used to identify breathing patterns or apnea. In addition, a variety of information can be derived via the communication components 1040, such as location via Internet Protocol (IP) geolocation, location via Wi-Fi® signal triangulation, or location via detecting an NFC beacon signal that can indicate a particular location, and so forth.

The various memories (e.g., memory 1004, main memory 1012, static memory 1014, and/or memory of the processors 1002) and/or storage unit 1016 can store one or more instructions or data structures (e.g., software) embodying or used by any one or more of the methodologies or functions described herein. These instructions (e.g., the instructions 1008), when executed by processors or processor circuitry, cause various operations to implement the embodiments discussed herein.

The instructions 1008 can be transmitted or received over the network 1020, using a transmission medium, via a network interface device (e.g., a network interface component included in the communication components 1040) and using any one of a number of well-known transfer protocols (e.g., hypertext transfer protocol (HTTP)). Similarly, the instructions 1008 can be transmitted or received using a transmission medium via the coupling 1026 (e.g., a peer-to-peer coupling) to the devices 1022.

To further illustrate the methods and apparatuses described herein, a non-limiting set of Example embodiments are set forth below as numerically identified Examples.

Example 1 includes a system comprising: an intraocular pressure (IOP) sensor, a blood pressure (BP) sensor, a patient treatment system, and a processor circuit configured to determine an ocular health index (OHI) value for a patient based on IOP information from the IOP sensor, BP information from the BP sensor, and at least one of information about a therapy provided to the patient by the patient treatment system or information about a patient response to a therapy provided by the patient treatment system.

In Example 2, the subject matter of Example 1 can optionally include the processor circuit configured to determine a disease progression based on a trend of the OHI value, and in response to the disease progression corresponding to a worsening disease state for the patient, the processor circuit can be configured to change or update a gauge pressure parameter of the therapy provided by the patient treatment system to the patient.

In Example 3, the subject matter of Examples 1-2 can optionally include the patient treatment system configured to receive the OHI value from the processor circuit and, in response to the OHI value meeting a specified threshold criteria, change a gauge pressure parameter of the therapy provided by the patient treatment system.

In Example 4, the subject matter of Example 3 can optionally include the patient treatment system configured to increase a frequency or duration or intensity of an applied gauge pressure therapy when the OHI value indicates a worsening ocular health status for the patient.

In Example 5, the subject matter of Examples 1-4 can optionally include a user interface configured to receive information about the OHI value from the processor circuit and display the OHI value.

In Example 6, the subject matter of Examples 1-5 can optionally include a patient interface coupled to at least one of the patient treatment system and the processor circuit and configured to receive qualitative or quantitative health status information from the patient, and the processor circuit can be configured to determine the OHI value for the patient using the received health status information from the patient.

In Example 7, the subject matter of Examples 1-6 can optionally include the patient treatment system comprising a goggle-based treatment system configured to intermittently apply a gauge pressure to the patient in coordination with instructions from the processor circuit.

In Example 8, the subject matter of Examples 1-7 can optionally include the patient treatment system comprising a tissue interface system configured to intermittently apply a gauge pressure to a skin tissue surface of the patient.

In Example 9, the subject matter of Examples 1-8 can optionally include the patient treatment system comprising a glaucoma treatment system configured to intermittently provide a glaucoma therapy to the patient, and wherein the processor circuit is configured to determine a glaucoma disease progression indication based on the OHI value.

In Example 10, the subject matter of Examples 1-9 can optionally include the patient treatment system comprising a drug delivery system, and the processor circuit can be configured to determine the OHI value based on information about a drug therapy provided to the patient using the drug delivery system.

In Example 11, the subject matter of Examples 1-10 can optionally include the IOP sensor comprising a contact lens configured to measure IOP information about the patient when the contact lens is worn by the patient.

In Example 12, the subject matter of Examples 1-11 can optionally include the processor circuit configured to determine a first ocular perfusion pressure (OPP) characteristic of the patient based on the IOP information and the BP information, and the processor circuit can optionally be further configured to determine the OHI value using the first OPP characteristic and the information about the therapy provided to the patient by the patient treatment system.

In Example 13, the subject matter of Examples 1-12 can optionally include the IOP sensor configured to measure IOP information about the patient over a first time interval, and the BP sensor configured to measure BP information about the patient over the first time interval, and the patient treatment system configured to record information about a patient therapy during the first time interval, and the processor circuit configured to determine the OHI value for the patient based on the IOP information measured over the first time interval, the BP information measured over the first time interval, and the information about the patient therapy during the first time interval.

In Example 14, the subject matter of Example 13 can optionally include the processor circuit configured to determine the OHI value for the patient based on a change in the IOP information about the patient over the first time interval.

In Example 15, the subject matter of Examples 1-14 can optionally include the patient treatment system configured to provide a nocturnal gauge pressure therapy to the patient.

In Example 16, the subject matter of Examples 1-15 can optionally include using the processor circuit to determine an updated OHI value for the patient at particular intervals, such as least every X days, where X is an integer number of days.

In Example 17, the subject matter of Examples 1-16 can optionally include the IOP sensor and the BP sensor configured to measure, substantially concurrently, respective IOP and BP information about the patient.

In Example 18, the subject matter of Examples 1-17 can optionally include the processor circuit configured to generate a treatment parameter, based on the OHI value, for use by the patient treatment system in providing a subsequent therapy to the patient.

In Example 19, the subject matter of Examples 1-18 can optionally include a remote patient monitoring system and a communication circuit communicatively coupled to the processor circuit and the remote patient monitoring system. In Example 19, the processor circuit can be configured to intermittently or periodically provide the OHI value to the remote patient monitoring system using the communication circuit.

In Example 20, the subject matter of Example 19 can optionally include, in response to receiving the OHI value, the remote patient monitoring system configured to provide instructions to the patient treatment system to update a treatment parameter.

In Example 21, the subject matter of Examples 19-20 can optionally include, in response to receiving the OHI value, the remote patient monitoring system configured to solicit, via a patient interface, patient-reported status information about one or more of a weight of the patient, a body-mass index (BMI) of the patient, a diet or diet change of the patient, a drug regimen of the patient, or a drug regimen adherence of the patient.

Example 22 is a non-transitory computer-readable storage medium, the computer-readable storage medium including instructions that, when executed by a processor circuit, cause the processor circuit to: receive information about a first intraocular pressure (IOP) characteristic of a patient from an IOP sensor, receive information about a first blood pressure (BP) characteristic of the patient from a blood pressure sensor, receive information about a first patient therapy from a patient treatment system, and determine a quantitative ocular health index for the patient using the received information about the first IOP characteristic, using the received information about the first BP characteristic, and using the received information about the first patient therapy from the patient treatment system.

In Example 23, the subject matter of Example 22 includes instructions that, when executed by the processor circuit, cause the processor circuit to provide the quantitative ocular health index to at least one of a remote patient monitoring system or a patient interface device. The instructions can further include instructions to display or communicate the quantitative ocular health index to a patient or caregiver.

In Example 24, the subject matter of Examples 22-23 can optionally include instructions to cause the processor circuit to receive the information about the first patient therapy, including instructions to cause the processor circuit to receive information about a gauge pressure therapy applied to eyes of the patient by a goggle-based treatment system.

In Example 25, the subject matter of Examples 22-24 can optionally include instructions to cause the processor circuit to receive the information about the first patient therapy, including instructions to cause the processor circuit to receive information about a gauge pressure therapy applied to a skin tissue surface area of the patient by a tissue interface system, wherein the skin tissue surface area excludes an eye or eye cavity of the patient.

In Example 26, the subject matter of Examples 22-25 can optionally include instructions to cause the processor circuit to receive the information about the first patient therapy, including instructions to receive, from an eye treatment system, therapy information about an ocular therapy provided by a goggle-based treatment system to the patient, and wherein the instructions to cause the processor circuit to determine the ocular health index include instructions to cause the processor to determine a quantitative glaucoma health index for the patient based on the information about the first IOP characteristic, the first BP characteristic, and the ocular therapy.

In Example 27, the subject matter of Examples 22-26 can optionally include instructions to cause the processor circuit to receive information about the first patient therapy, including instructions to receive information about a drug therapy provided to the patient from a drug delivery system.

In Example 28, the subject matter of Examples 22-27 can optionally include instructions to cause the processor circuit to determine the ocular health index for the patient, including instructions to cause the processor circuit to determine a first ocular perfusion pressure (OPP) characteristic of the patient based on the first IOP and the first BP characteristics, and determine the ocular health index based on the first OPP and the information about the first patient therapy.

In Example 29, the subject matter of Examples 22-28 can optionally include instructions to configure the processor circuit to: receive information about a subsequent second IOP characteristic of the patient, receive information about a subsequent second BP characteristic of the patient, and determine the ocular health index using information about a difference between the first and second IOP characteristics and using information about a difference between the first and second BP characteristics.

In Example 30, the subject matter of Example 29 can optionally include information about the first IOP characteristic corresponding to a first IOP measurement event and the information about the second IOP characteristic corresponding to a subsequent second IOP measurement event, and wherein the instructions to configure the processor circuit to receive information about the first patient therapy include instructions to configure the processor circuit to receive information about a therapy provided to the patient between the first and second IOP measurement events.

In Example 31, the subject matter of Examples 29-30 can optionally include the information about the first and second IOP characteristics received over one or more nocturnal monitoring periods.

In Example 32, the subject matter of Examples 29-31 can optionally include the information about the first and second IOP characteristics received over one or more diurnal monitoring periods.

In Example 33, the subject matter of Examples 22-32 can optionally include the information about the first IOP characteristic indicating IOP information about the patient over a first interval, the information about the first BP characteristic indicating BP information about the patient over the same first interval, and the information about the first patient therapy indicating therapy information corresponding to the same first interval.

In Example 34, the subject matter of Examples 22-33 can optionally include instructions further configuring the processor circuit to receive, from a patient interface, information about a patient-reported field of vision, and the instructions to configure the processor circuit to determine the ocular health index for the patient can include using the information about the patient-reported field of vision.

In Example 35, the subject matter of Examples 22-34 can optionally include instructions further configuring the processor circuit to measure the first IOP characteristic and the first BP characteristic of the patient substantially concurrently.

In Example 36, the subject matter of Examples 22-35 can optionally include instructions further configuring the processor circuit to generate a treatment recommendation for the patient based on the ocular health index.

In Example 37, the subject matter of Examples 22-36 can optionally include instructions further configuring the processor circuit to update a treatment parameter used by the patient treatment system to provide subsequent therapy to the patient.

In Example 38, the subject matter of Example 37 can optionally include instructions to update the treatment parameter including instructions to update a glaucoma therapy frequency or glaucoma therapy duration provided to the patient using a goggle-based treatment system.

In Example 39, the subject matter of Examples 22-38 can optionally include instructions further configuring the processor circuit to receive, from a patient interface, patient-reported status information about one or more of a weight of the patient, a body-mass index (BMI) of the patient, a diet or diet change of the patient, a drug regimen of the patient, or a drug regimen adherence of the patient. In Example 39, the instructions to configure the processor circuit to determine the ocular health index for the patient can include instructions to use the patient-reported status information to determine the ocular health index.

In Example 40, the subject matter of Examples 22-39 can optionally include instructions further configuring the processor circuit to receive, from a remote patient monitoring system and in response to information about the ocular health index, instructions to enable the patient treatment system to begin a new therapy for the patient, instructions to continue a previous therapy for the patient, or instructions to inhibit provision of a therapy to the patient.

In Example 41, the subject matter of Examples 22-40 can optionally include instructions further configuring the processor circuit to solicit patient-reported status information about one or more of a weight of the patient, a body-mass index (BMI) of the patient, a diet or diet change of the patient, a drug regimen of the patient, or a drug regimen adherence of the patient.

Example 42 can include a method for evaluating a patient status associated with an eye health of a patient, the method comprising: receiving information about a first intraocular pressure (IOP) characteristic of the patient, receiving information about a first blood pressure (BP) characteristic of the patient, receiving information about a first patient therapy from a patient treatment system, and determining a quantitative ocular health index for the patient using the received information about the first IOP characteristic, using the received information about the first BP characteristic, and using the received information about the first patient therapy from the patient treatment system. Example 42 can further include providing the quantitative ocular health index to at least one of a remote patient monitoring system or a patient interface device and using the remote patient monitoring system or the patient interface device to display the quantitative ocular health index.

In Example 43, the subject matter of Example 42 can optionally include receiving the information about the first patient therapy includes receiving information about a gauge pressure therapy applied to the patient by a treatment system.

In Example 44, the subject matter of Example 43 can optionally include receiving the information about the gauge pressure therapy includes receiving information about a gauge pressure therapy applied to eyes of the patient by a goggle-based treatment system.

In Example 45, the subject matter of Examples 43-44 can optionally include receiving the information about the gauge pressure therapy including information about a gauge pressure therapy applied to a skin tissue surface area of the patient by a tissue interface system, and the skin tissue surface area excludes an eye or eye cavity of the patient.

In Example 46, the subject matter of Examples 43-45 can optionally include receiving the information about the first patient therapy including receiving, from an eye treatment system, therapy information about an ocular therapy provided by the system to the patient. In Example 46, determining the ocular health index can include determining a quantitative glaucoma health index for the patient based on the information about the first IOP characteristic, the first BP characteristic, and the ocular therapy.

In Example 47, the subject matter of Examples 42-46 can optionally include determining the ocular health index including determining an optical health of the patient.

In Example 48, the subject matter of Examples 42-47 can optionally include receiving information about the first patient therapy including receiving information about a drug therapy provided to the patient from a drug delivery system.

In Example 49, the subject matter of Examples 42-48 can optionally include determining the ocular health index for the patient including determining a first ocular perfusion pressure (OPP) characteristic of the patient based on the first IOP and the first BP characteristics, and determining the ocular health index based on the first OPP and the information about the first patient therapy.

In Example 50, the subject matter of Examples 42-49 can optionally include determining the ocular health index using information about a cerebrospinal fluid pressure characteristic of the patient.

In Example 51, the subject matter of Examples 42-50 can optionally include receiving the information about the first BP characteristic including receiving the blood pressure information from a sensor on a contact lens worn by the patient.

In Example 52, the subject matter of Examples 42-51 can optionally include receiving information about a subsequent second IOP characteristic of the patient, receiving information about a subsequent second BP characteristic of the patient, and determining the ocular health index using information about a difference between the first and second IOP characteristics and using information about a difference between the first and second BP characteristics.

In Example 53, the subject matter of Example 52 can optionally include the information about the first IOP characteristic corresponds to a first IOP measurement event and the information about the second IOP characteristic corresponds to a subsequent second IOP measurement event, and receiving information about the first patient therapy includes receiving information about a therapy provided to the patient between the first and second IOP measurement events.

In Example 54, the subject matter of Examples 52-53 can optionally include receiving the information about the first and second IOP characteristics including receiving the information over one or more nocturnal monitoring periods.

In Example 55, the subject matter of Examples 52-54 can optionally include receiving the information about the first and second IOP characteristics including receiving the information over one or more diurnal monitoring periods.

In Example 56, the subject matter of Examples 52-55 can optionally include determining the ocular health index for the patient periodically or intermittently, such as every X number of days, where X is an integer.

In Example 57, the subject matter of Examples 42-56 can optionally include the information about the first IOP characteristic indicating IOP information about the patient over a first interval, the information about the first BP characteristic indicating BP information about the patient over the same first interval, and the information about the first patient therapy indicating therapy information corresponding to the same first interval.

In Example 58, the subject matter of Examples 42-57 can optionally include receiving, from a patient interface, information about a patient-reported field of vision, and determining the ocular health index for the patient using the information about the patient-reported field of vision.

In Example 59, the subject matter of Examples 42-58 can optionally include measuring the first IOP characteristic and the first BP characteristic of the patient substantially concurrently.

In Example 60, the subject matter of Examples 42-59 can optionally include generating a treatment recommendation for the patient based on the ocular health index, wherein the treatment recommendation can be implemented at least in part by the patient treatment system.

In Example 61, the subject matter of Examples 42-60 can optionally include updating a treatment parameter used by the patient treatment system to provide subsequent therapy to the patient.

In Example 62, the subject matter of Example 61 can optionally include updating the treatment parameter including updating a glaucoma therapy frequency or glaucoma therapy duration for the patient.

In Example 63, the subject matter of Examples 42-62 can optionally include communicating a therapy plan to the patient based on the ocular health index, wherein the therapy plan includes or uses the patient treatment system or one or more sensors coupled thereto.

In Example 64, the subject matter of Examples 42-63 can optionally include receiving, from a patient interface, patient-reported status information about one or more of a weight of the patient, a body-mass index (BMI) of the patient, a diet or diet change of the patient, a drug regimen of the patient, or a drug regimen adherence of the patient, and determining the ocular health index for the patient includes using the patient-reported status information.

In Example 65, the subject matter of Examples 42-64 can optionally include automatically reporting the ocular health index to a remote patient monitoring system.

In Example 66, the subject matter of Example 65 can optionally include receiving, from the remote patient monitoring system and in response to the reported ocular health index, instructions to enable the patient treatment system to begin a new therapy for the patient, instructions to continue a previous therapy for the patient, or instructions to inhibit provision of a therapy to the patient.

In Example 67, the subject matter of Examples 65-66 can optionally include, in response to the reported ocular health index, soliciting patient-reported status information about one or more of a weight of the patient, a body-mass index (BMI) of the patient, a diet or diet change of the patient, a drug regimen of the patient, or a drug regimen adherence of the patient.

Example 68 is at least one machine-readable medium including instructions that, when executed by processing circuitry, cause the processing circuitry to perform operations to implement of any of Examples 1-21 or 42-67.

Example 69 is an apparatus comprising means to implement of any of Examples 1-67.

Example 70 is a system to implement of any of Examples 1-67.

Each of these non-limiting examples can stand on its own, or can be combined in various permutations or combinations with one or more of the other examples.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-67. (canceled)

68. A patient treatment system to determine an ocular health index (OHI) value for a patient eye, the patient treatment system comprising:
a processor circuit, in communication with a pump, the processor circuit configured to:
receive a first indication of intraocular pressure (IOP) in the patient eye over a first time interval and a first indication of blood pressure (BP) from the patient over the first time interval; and
generate the OHI value based at least in part on the first indication of IOP and the first indication of BP.

69. The patient treatment system of claim 68, wherein the processor circuit is configured to determine an indication of glaucoma disease progression based on the OHI value.

70. The patient treatment system of claim 68, wherein the OHI value is an indication of ocular perfusion pressure (OPP).

71. The patient treatment system of claim 69, wherein the processor circuit is configured to:
receive the first indication of intraocular pressure (IOP) in the patient eye over the first time interval and the first indication of a blood pressure (BP) from the patient over the first time interval, wherein the first time interval occurs prior to applying a therapy to the patient eye;
receive a second indication of IOP in the patient eye over a second time interval and a second indication of BP from the patient over the second time interval, wherein the second time interval occurs after applying the therapy to the patient eye;
determine an IOP difference between the received first and second indications of IOP;
determine a BP difference between the received first and second indications of BP; and
generate the OHI value based at least in part on the determined IOP difference and the determined BP difference to determine an effect of the therapy on the patient eye.

72. The patient treatment system of claim 71, wherein the processor circuit is configured to determine the OHI value based at least in part on information about the therapy provided to the patient.

73. The patient treatment system of claim 71, wherein the therapy applied to the patient eye includes at least one of a goggle-based therapy or a drug delivery therapy.

74. The patient treatment system of claim 73, wherein the therapy applied to the patient eye is the goggle-based therapy.

75. The patient treatment system of claim 74, wherein the processor circuit is configured to determine the OHI value based at least in part on information about the goggle-based therapy provided to the patient.

76. The patient treatment system of claim 73, wherein the therapy applied to the patient eye is the drug delivery therapy.

77. The patient treatment system of claim 68, further comprising a patient interface configured to identify a field of vision of the patient wherein the patient interface includes at least one of a virtual reality (VR) headset or other head-mounted system.

78. The patient treatment system of claim 68, further comprising:
a goggle, configured to fit over the patient eye to form a cavity over the patient eye; and
a pump, in communication with the cavity, configured to affect establish a fluid pressure in the cavity.

79. A method of using a patient treatment system to determine an ocular health index (OHI) value for a patient eye, the method comprising:
receiving with a processor circuit a first indication of intraocular pressure (IOP) in the patient eye over a first time interval and a first indication of blood pressure (BP) from the patient over the first time interval; and
generating the OHI value based at least in part on the first indication of IOP and the first indication of BP.

80. The method of claim 79, wherein generating the OHI value includes generating an indication of glaucoma disease progression based on the OHI value.

81. The method of claim 79, wherein generating the OHI value includes generating an indication of ocular perfusion pressure (OPP).

82. The method of claim 79, wherein,
receiving with a processor circuit includes receiving the first indication of intraocular pressure (IOP) in the patient eye over the first time interval and the first indication of a blood pressure (BP) from the patient over the first time interval, wherein the first time interval occurs prior to applying a therapy to the patient eye; and,
the method further comprising:
receiving a second indication of IOP in the patient eye over a second time interval and a second indication of BP from the patient over the second time interval, wherein the second time interval occurs after applying the therapy to the patient eye;
determining an IOP difference between the received first and second indications of IOP;
determining a BP difference between the received first and second indications of BP, and
generating the OHI value based at least in part on the determined IOP difference and the determined BP difference to determine an effect of the therapy on the patient eye.

83. The method of claim 82, wherein generating the OHI value includes generating the OHI value based at least in part on information about the therapy provided to the patient.

84. The method of claim 82, wherein generating the OHI value includes generating the OHI value with a therapy applied to the patient eye, the therapy including at least one of a goggle-based therapy or a drug delivery therapy.

85. The method of claim 84 wherein generating the OHI value includes generating the OHI value with the goggle-based therapy.

86. The method of claim 84 wherein generating the OHI value includes generating the OHI value with the drug delivery therapy.

87. The method of claim 79 further comprising receiving information from a patient interface configured to identify a field of vision of the patient to determine or update the OHI value.
